(19) United States
(12) Patent Application Publication
(10) Pub. No.: US 2024/0115282 A1
(43) Pub. Date: Apr. 11, 2024

(54) SURGICAL CONSTRICTING DEVICES AND SURGICAL METHODS UTILIZING SAME

(71) Applicant: Covidien LP, Mansfield, MA (US)

(72) Inventors: Jacob C. Baril, Norwalk, CT (US); Garrett P. Ebersole, Hamden, CT (US)

(21) Appl. No.: 17/963,359

(22) Filed: Oct. 11, 2022

(51) Int. Cl. A61B 17/29

(52) U.S. Cl. CPC A61B 17/29; A61B 2017/2902; A61B 2017/2906; A61B 2017/2946

(57) ABSTRACT

A surgical constricting device includes an elongated body, a first grasping pad, and a second grasping pad. The elongated body includes a central core and an outer sheath surrounding the central core. The central core is made from a first material, and the outer sheath is made from a second material. The elongated body is configured to maintain its position when wrapped around itself. The first grasping pad is disposed at a first end of the elongated body and is configured to be engaged by a surgical instrument. The second grasping pad is disposed at a second end of the elongated body and is configured to be engaged by a surgical instrument.

### BACKGROUND

This disclosure relates to surgical instruments and surgical methods utilizing the same. More particularly, this disclosure relates to surgical constricting devices and to methods for deploying the same onto tissue.

Surgical graspers are known in the art and are used for a number of distinct and useful surgical procedures including endoscopic, open, and robotic surgical procedures. Surgical graspers having various sizes (e.g., diameters) are configured to manipulate tissue and/or surgical fasteners (e.g., surgical clips) or implants, for instance, within the body cavity. Such surgical clips are typically fabricated from a biocompatible material and are usually compressed over tissue. Once applied to tissue, the compressed surgical clip terminates the flow of fluid through the tissue.

Certain surgical procedures or situations may benefit from the deployment of a surgical constricting device to crimp a vessel, or to retain a catheter in a vessel without occluding the vessel (during cholangiograms, for instance).

### SUMMARY

This disclosure relates to a surgical constricting device that includes an elongated body, a first grasping pad, and a second grasping pad. The elongated body includes a central core and an outer sheath surrounding the central core. The central core is made from a first material, and the outer sheath is made from a second material. The elongated body is configured to maintain its position when wrapped around itself. The first grasping pad is disposed at a first end of the elongated body and is configured to be engaged by a surgical instrument. The second grasping pad is disposed at a second end of the elongated body and is configured to be engaged by a surgical instrument.

In disclosed embodiments, the central core defines a circular cross-section. It is also disclosed that the circular cross-section of the central core defines a diameter of between about 0.035 inches and about 0.065 inches. It is also disclosed that the outer sheath defines a ring-like cross-section. It is further disclosed that the outer sheath defines a thickness of between about 0.09375 inches and about 0.125 inches.

In disclosed embodiments, the first material is stainless steel and the second material is selected from the group consisting of polyurethane, polydimethylsiloxane, polypropylene, polyethylene, and a medium durometer silicone.

In disclosed embodiments, a length of the surgical constricting device is between about 1.75 inches and about 2.5 inches.

In disclosed embodiments, the elongated body portion is generally cylindrical. It is also disclosed that each of the first grasping pad and the second grasping pad is in the general shape of a rectangular prism.

This disclosure also relates to a surgical constricting device that includes an elongated body including a central core and an outer sheath surrounding the central core. The central core is made from stainless steel, and the outer sheath is made from a second material selected from the group consisting of polyurethane, polydimethylsiloxane, polypropylene, polyethylene, and a medium durometer silicone. The elongated body is configured to maintain its position when wrapped around itself.

In disclosed embodiments, the central core defines a circular cross-section. It is also disclosed that the circular cross-section of the central core defines a diameter of between about 0.035 inches and about 0.065 inches.

This disclosure also relates to a method of fixing a catheter to a vessel. The method includes: positioning a surgical constricting device such that a central portion of an elongated body of the surgical constricting device is positioned adjacent the vessel, and a first end and a second end of the surgical constricting device are positioned proximally of the vessel; grasping the first end and the second end of the surgical constricting device with an end effector of a surgical grasping instrument; and rotating the end effector about a longitudinal axis defined by a shaft of the surgical grasping instrument relative to the vessel to create a wrapped portion of the surgical constricting device.

In disclosed embodiments, rotating the end effector includes rotating the end effector more than 360°.

In disclosed embodiment, the method includes removing the surgical grasping instrument from engagement with the surgical constricting device without impacting the wrapped portion of the surgical constricting device.

### BRIEF DESCRIPTION OF THE DRAWINGS

Embodiments of the disclosure are described in detail with reference to the drawing figures wherein like reference numerals identify similar or identical structural elements and:

FIG. 1 is a plan view of a surgical constricting device in accordance with embodiments of the disclosure;

FIG. 2 is a perspective, partial, transverse, cross-sectional view of the surgical constricting device of FIG. 1;

FIG. 3 is a perspective view of an end portion of the surgical constricting device of FIG. 1;

FIG. 4 is a perspective view of a robotic surgical instrument including an end effector positioned within a patient and adjacent a vessel;

FIG. 5 is a perspective view of the end effector of the robotic surgical instrument of FIG. 4 illustrated adjacent the surgical constricting device of FIG. 1, which is partially surrounding the vessel;

FIG. 6 is a perspective view of the end effector of the robotic surgical instrument of FIG. 4 grabbing the surgical constricting device of FIG. 1, which is partially surrounding the vessel;

FIG. 7 is a perspective view of the end effector of the robotic surgical instrument of FIG. 4 grabbing and twisting the surgical constricting device of FIG. 1, which is surrounding the vessel;

FIG. 8 is a perspective view of the surgical constricting device of FIG. 1 partially constricting the vessel; and

FIG. 9 is a schematic illustration of a robotic surgical system configured for use in accordance with the disclosure.

### DETAILED DESCRIPTION

Embodiments of the disclosed surgical constricting device are described in detail with reference to the drawings, in which like reference numerals designate identical or corresponding elements in each of the several views. Non-limiting examples of surgical instruments according to the disclosure include manual, robotic, mechanical and/or electromechanical, and the like. As used herein the term “distal” refers to that portion of the surgical instrument, or component thereof, farther from the user, while the term “proximal” refers to that portion of the surgical instrument, or component thereof, closer to the user.

As will be described in greater detail below, the disclosure includes a surgical constricting device, and a surgical instrument for employing the surgical constricting device to fix a catheter to a vessel, for instance. Related methods of use are also encompassed by this disclosure.

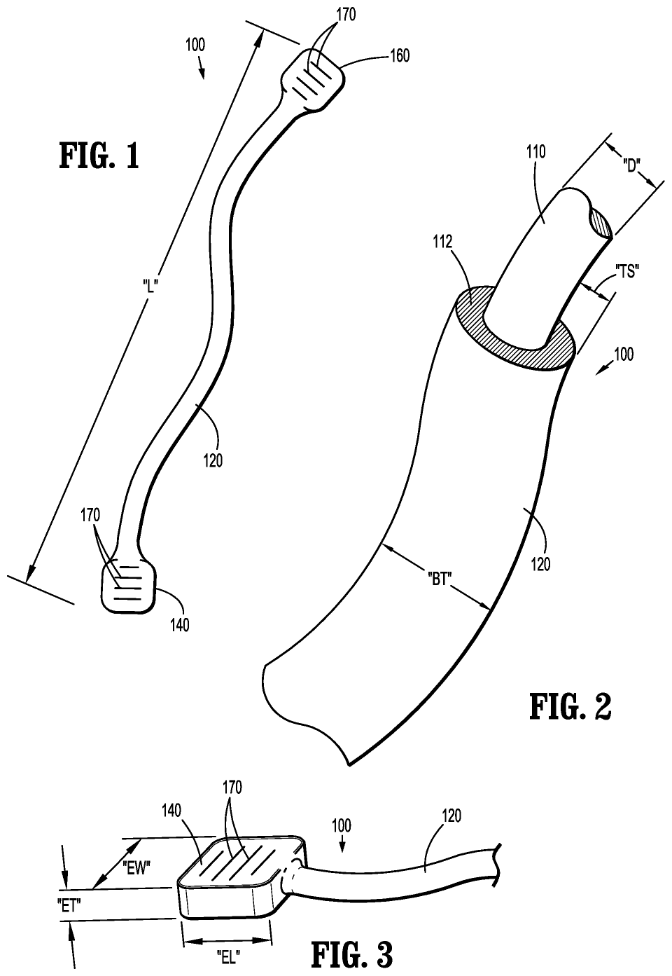

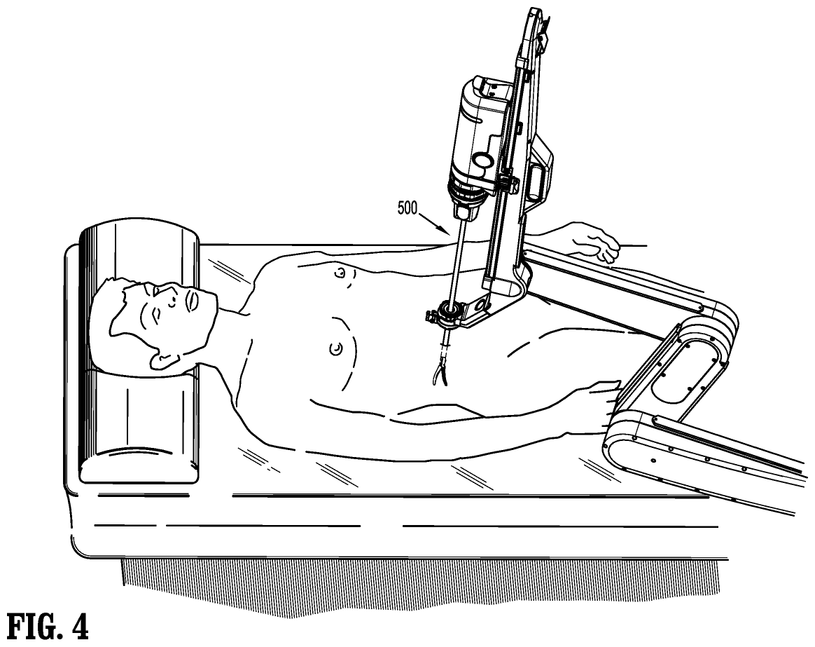

FIGS. 1-9 illustrate a surgical constricting device 100 and a surgical grasping instrument 500 for fastening or securing the surgical constricting device 100 to tissue in accordance with embodiments of the disclosure. While the figures illustrate a robotic surgical grasper (e.g., FIG. 4), other types of surgical graspers (e.g., open and endoscopic surgical graspers, handheld and hand-powered graspers, and handheld and battery powered graspers, etc.) are encompassed by the scope of the present disclosure and are usable with the disclosed surgical constricting device 100.

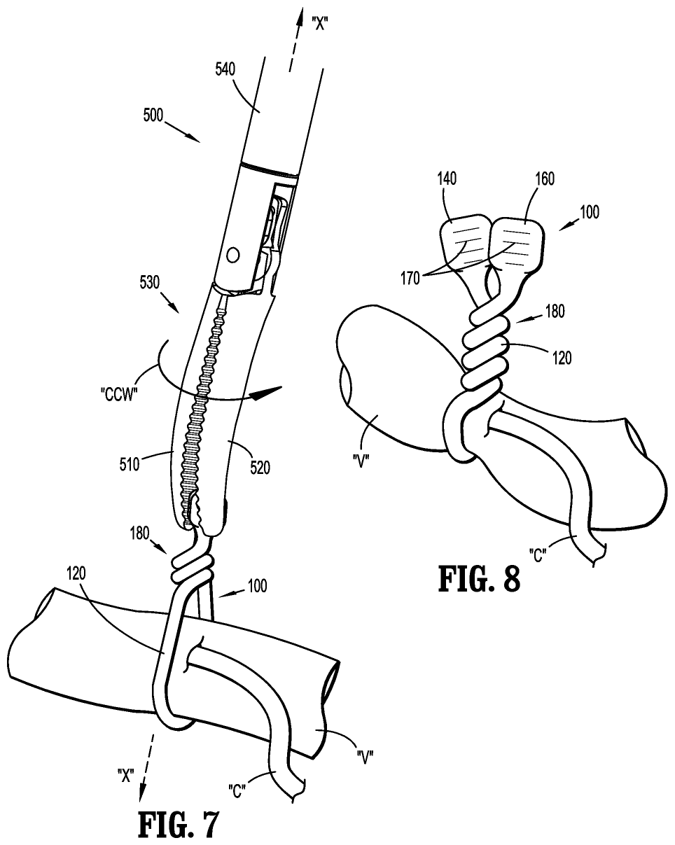

With initial reference to FIG. 1, the surgical constricting device 100 includes an elongated body 120, a first grasping pad or first end 140, and a second grasping pad or second end 160. The elongated body 120 extends between and interconnects the first end 140 and the second end 160. Generally, the elongated body 120 has enough flexibility to be wrapped around a vessel “V” and twisted multiple times (see FIGS. 7 and 8, for instance). Additionally, the elongated body 120 has enough rigidity to be wrapped around itself multiple times while maintaining that position, i.e., without unwinding (see FIG. 8, for instance).

With continued reference to FIG. 1, the surgical constricting device 100 defines a length “L.” In embodiments, the length “L” is between about 1.75 inches and about 2.5 inches, which is sufficient to grasp and occlude most vessels. If larger or smaller structures are desired to be occluded, a surgical constricting device 100 having a longer or short length “L,” respectively, can be used. The surgical constricting device 100 having a length “L” in this range enables the surgical constricting device 100 to be positioned around a target vessel “V” and wrapped a sufficient number of times around itself to maintain securement of the surgical constricting device 100 to the vessel “V,” as shown in FIG. 8, for example.

Referring now to FIG. 2, the surgical constricting device 100 includes a central core 110 and an outer sheath 112. The material, size and/or shape of the central core 110 enable the central core 110 to be wrapped around a vessel “V” and twisted around itself while maintaining its twisted shape, or any other shape created by the surgeon. In embodiments, the central core 110 is made from stainless steel wire or another suitable material that is bio-inert and holds its shape when being bent into a configuration. In the embodiment illustrated in FIG. 2, the cross-section of the central core 110 is a circle, but cross-sections having other regular or irregular shapes are encompassed by the disclosure. In embodiments where the central core 110 has a circular cross-section, the diameter “D” of the central core may be between about 0.035 inches and about 0.065 inches, e.g., equal to about 0.050 inches, thereby equaling a cross-sectional area of between about 0.001 inches and about 0.003 inches, e.g., equal to about 0.002 inches. In embodiments where the central core 110 does not have a circular cross-section, the cross-sectional area may be similar or equal to the cross-sectional area of the central core 110 having a circular cross-section, as discussed above. A single central core having varying cross-sections is also envisioned.

Further the central core 110 may extend through an entirety of the surgical constricting device 100, through only the elongated body 120, or through only portions of the elongated body 120. For example, in embodiments, the central core 110 extends through portions of the elongated body 120 but does not extend through the middle third (approximately) of the elongated body 120 such that the portion of the elongated body 120 that contacts the vessel “V” is free from having a central core 110, thereby minimizing undue pressure or strain on the vessel “V” during certain procedures, for instance.

With continued reference to FIG. 2, the outer sheath 112 radially surrounds the central core 110. In embodiments, the outer sheath 112 is made from polyurethane, polydimethylsiloxane, polypropylene, polyethylene, a medium durometer silicone, or another suitable material. It is further envisioned that the outer sheath 112 may be embedded with a medicament, such as include non-steroidal anti-inflammatory drugs (NSAIDs), other anti-inflammatory agents, anti-microbials, anti-bacterials, local anesthetics, or other suitable medicaments. In the embodiment illustrated in FIG. 2, the outer sheath 112 has a circular or ring-like cross-section, but cross-sections having other regular or irregular shapes are encompassed by the disclosure. Additionally, while the outer sheath 112 is shown having the same cross-section shape as the central core 110 (i.e., circular), the disclosure encompasses the cross-section of the outer sheath 112 being a different shape than the cross-section of the central core 110. Further, the outer sheath 112 defines a thickness “TS” (FIG. 2) of between about 0.014 inches and about 0.045 inches, e.g., equal to about 0.03 inches.

In embodiments, the thickness “TS” of the outer sheath 112 may be consistent throughout the surgical constricting device 100, may be consistent along the elongated body 120 and have a different value at the first end 140 and/or the second end 160, or may vary along the elongated body 120. For instance, in embodiments, the thickness “TS” of the outer sheath 112 may be less along the middle third of the elongated body 120 than the remainder of the surgical constricting device 100 such that the portion of the elongated body 120 that contacts the vessel “V” has more flexibility. Alternatively, the thickness “TS” of the outer sheath 112 may be greater along the middle third of the elongated body 120 than the remainder of the surgical constricting device 100 such that the portion of the elongated body 120 that contacts the vessel “V” has more padding. In this manner undue pressure or strain on the vessel “V” may be minimized during certain procedures, for instance.

In the embodiment illustrated in FIGS. 1 and 3, for example, the shape of the surgical constricting device 100 (including the shape of the outer sheath 112), as viewed from the top and from the side, for instance, is different at the first end 140 and the second end 160 than at the elongated body 120. More particularly, while the elongated body 120 may generally define a cylindrical shape (including curves when positioned as shown in FIG. 1), each of the first end 140 and the second end 160 may generally be in the shape of a rectangular prism with curved edges, as shown in FIGS. 1 and 3, defining an end length “EL,” an end width “EW,” and an end thickness “ET.” It is envisioned that other shapes for the first end 140 and the second end 160 are within the scope of the disclosure, including and not limited to circular, ovular, triangular, rings/loops, etc.

Additionally, the elongated body 120 defines a body thickness “BT” equal to the diameter “D” of the inner core 110 plus twice the thickness “TS” of the outer sheath 112 (FIG. 2). In embodiments, the body thickness “BT” of the elongated body is between about 0.09375 inches and about 0.125 inches. While the body thickness “BT” of the elongated body 120 is shown in FIG. 3 as being slightly smaller than the end thickness “ET” of the first end 140, embodiments are also disclosed where the first end 140 and/or the second end 160 have a smaller, equal, or substantially equal end thickness “ET” than the body thickness “BT.”

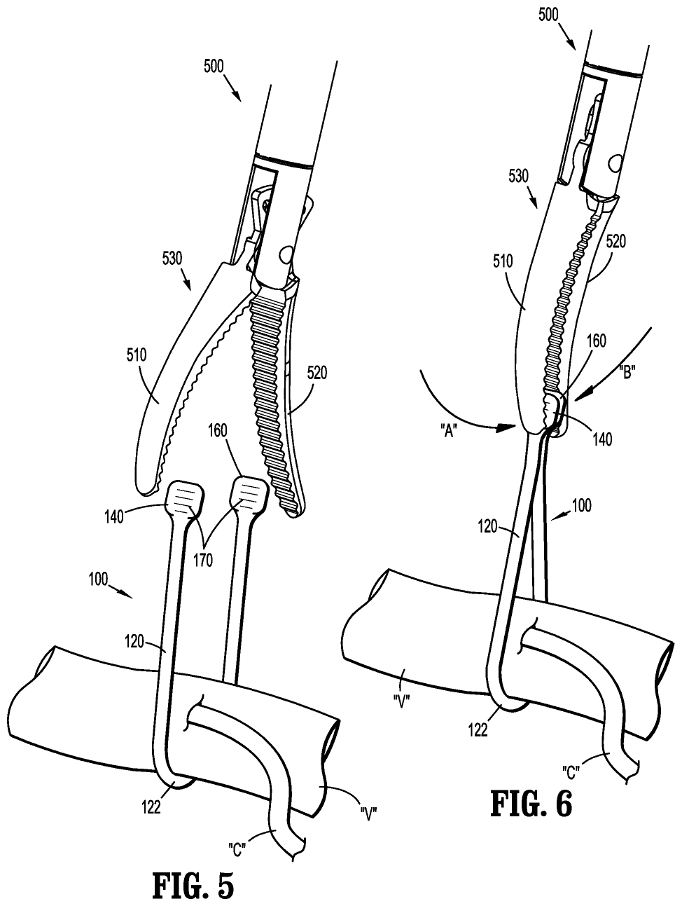

Further, and with continued reference to FIGS. 1 and 3, the first end 140 and the second end 160 include a plurality of ribs 170 thereon. The ribs 170 are raised portions that are configured to help the surgical grasping instrument 500 achieve an optimal or enhanced grip on the surgical constricting device 100, for instance. Moreover, the disclosure encompasses an underside of the first end 140 and the second end 160 also including the plurality of ribs 170 (see FIGS. 5 and 8, for example). Other grip enhancing features are contemplated, including and not limited to, knurling, dimpling, and the like.

Referring now to FIGS. 5-8, when a user desires to place the surgical constricting device 100 onto tissue or a vessel “V” and/or a catheter “C,” for instance, (e.g., to variably occlude a vessel, or to retain a catheter in a vessel without occluding the vessel such as during cholangiograms), the user initially maneuvers the surgical constricting device 100 into a U-like shape and positions a central portion 122 of the elongated body 120 of the surgical constricting device 100 adjacent the vessel “V” (see FIG. 5). This maneuvering and/or positioning of the surgical constricting device 100 can be performed by the surgical grasping instrument 500, a different type of instrument, or by hand, for example.

Next, at least one of a first jaw 510 or a second jaw 520 of an end effector 530 of the surgical grasping instrument 500 is moved toward the other (in the general directions of arrows “A” and “B,” respectively, in FIG. 6) into a closed or approximated position such that the first end 120 and the second end 140 of the surgical constricting device 100 contact each other (FIG. 6). It is contemplated that an end effector of a second surgical instrument (not shown) may be used to facilitate placing of the first end 120 and the second end 140 of the surgical constricting device 100 between the first jaw 510 and the second jaw 520 of end effector 530.

After the first jaw 510 and the second jaw 520 are in the closed position, the end effector 530 is rotated in the general direction of arrow “CCW” (FIG. 7), or the opposite direction, about a longitudinal axis “X” defined by a shaft 540 of the surgical grasping instrument 500, and relative to the vessel “V.” This rotation of the end effector 530 causes portions of the surgical constricting device 100 to wrap around other portions of the surgical constricting device 100, thereby creating a wrapped portion 180 (FIGS. 7 and 8). The more the surgical grasping instrument 500 is rotated, the larger the wrapped portion 180 becomes. Moreover, since certain surgical grasping instruments 500 (e.g., robotic instruments) can be easily rotated more than 360°, the use of such an instrument can quickly and easily create a desired wrapped portion 180. Further, the number of rotations of the end effector 530 of the surgical grasping instrument 500 (and the size of the wrapped portion 180, for instance) may directly impact the amount of pressure or constriction produced by the surgical constricting device 100 on the vessel “V.” Thus, a user can adjust or fine tune the amount of constriction of the vessel “V” by rotating the end effector 530 in a first direction (e.g., counter-clockwise, in the general direction of arrow “CCW”) to increase the amount of constriction or in a second, opposite direction (e.g., clockwise) to decrease the amount of constriction.

After the surgical constricting device 100 is properly positioned, a surgical procedure, such as a cholangiogram, may be performed. When the surgical procedure is complete, for instance, the surgical constricting device 100 may be removed from the vessel “V” and/or the catheter “C.” To remove the surgical constricting device 100 from the vessel “V” and/or the catheter “C”, the user may use the surgical grasping device 500 to unwind the surgical constricting device 100, and then use the surgical grasping device 500 to grab either the first end 140 or the second end 160, and remove the surgical constricting device 100 from a body cavity, for instance.

Alternatively, to remove the surgical constricting device 100 from the vessel “V” and/or the catheter “C”, an alternate device may be used, such as, for example, surgical shears (which are robotically or hand controlled), which is used to sever or cut elongate body 120 (e.g., the central portion 122 of elongate body 120) and thus release the vessel “V” and/or the catheter “C” from the surgical constricting device 100.

The various embodiments disclosed herein may also be configured to work with robotic surgical systems and what is commonly referred to as “Telesurgery.” Such systems employ various robotic elements to assist the surgeon in the operating theater and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include, remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prepare the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

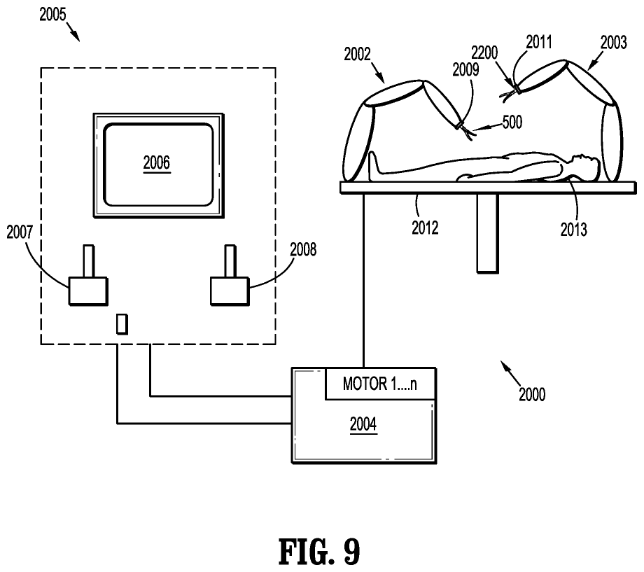

With reference to FIG. 9, a surgical system, such as, for example, a robotic surgical system is shown generally as surgical system 2000 and is usable with the surgical constricting device 100 and/or the surgical grasping device 500, or portions thereof, of the disclosure. Surgical system 2000 generally includes a plurality of robotic arms 2002, 2003, a control device 2004, and an operating console 2005 coupled with control device 2004. Operating console 2005 includes a display device 2006, which is set up in particular to display three-dimensional images; and manual input devices 2007, 2008, by means of which a person (not shown), for example a surgeon, is able to telemanipulate robotic arms 2002, 2003 in a first operating mode, as known in principle to a person skilled in the art.

Each of the robotic arms 2002, 2003 is composed of a plurality of members, which are connected through joints. System 2000 also includes an instrument drive unit 2200 connected to distal ends of each of robotic arms 2002, 2003. The surgical grasping device 500, or portions thereof, may be attached to the instrument drive unit 2200, in accordance with any one of several embodiments disclosed herein, as will be described in greater detail below.

Robotic arms 2002, 2003 may be driven by electric drives (not shown) that are connected to control device 2004. Control device 2004 (e.g., a computer) is set up to activate the drives, in particular by means of a computer program, in such a way that robotic arms 2002, 2003, their instrument drive units 2200 and thus the surgical grasping device 500 (including the end effector 530) execute a desired movement according to a movement defined by means of manual input devices 2007, 2008. Control device 2004 may also be set up in such a way that it regulates the movement of robotic arms 2002, 2003 and/or of the drives.

Surgical system 2000 is configured for use on a patient 2013 lying on a patient table 2012 to be treated in a minimally invasive manner by means of the surgical clip applier 100. Surgical system 2000 may also include more than two robotic arms 2002, 2003, the additional robotic arms likewise being connected to control device 2004 and being telemanipulatable by means of operating console 2005.

Reference may be made to U.S. Pat. No. 8,828,023, entitled “Medical Workstation,” the entire content of which is incorporated herein by reference, for a detailed discussion of the construction and operation of surgical system 2000.

It should be understood that the foregoing description is only illustrative of the disclosure. Various alternatives and modifications can be devised by those skilled in the art without departing from the disclosure. Accordingly, this disclosure is intended to embrace all such alternatives, modifications and variances. The embodiments described with reference to the attached drawing figures are presented only to demonstrate certain examples of the disclosure. Other elements, steps, methods and techniques that are insubstantially different from those described above and/or in the appended claims are also intended to be within the scope of the disclosure.

What is claimed is:

1. A surgical constricting device, comprising:
an elongated body including a central core and an outer sheath surrounding the central core, the central core made from a first material, the outer sheath made from a second material, the elongated body configured to maintain its position when wrapped around itself;
a first grasping pad disposed at a first end of the elongated body and configured to be engaged by a surgical instrument; and
a second grasping pad disposed at a second end of the elongated body and configured to be engaged by a surgical instrument.

2. The surgical constricting device according to claim 1, wherein the central core defines a circular cross-section.

3. The surgical constricting device according to claim 2, wherein the circular cross-section of the central core defines a diameter of between about 0.035 inches and about 0.065 inches.

4. The surgical constricting device according to claim 2, wherein the outer sheath defines a ring-like cross-section.

5. The surgical constricting device according to claim 3, wherein the outer sheath defines a thickness of between about 0.014 inches and about 0.045 inches.

6. The surgical constricting device according to claim 1, wherein the first material is stainless steel.

7. The surgical constricting device according to claim 6, wherein the second material is selected from the group consisting of polyurethane, polydimethylsiloxane, polypropylene, polyethylene, and a medium durometer silicone.

8. The surgical constricting device according to claim 6, wherein the second material is medium durometer silicone.

9. The surgical constricting device according to claim 1, wherein the second material is medium durometer silicone.

10. The surgical constricting device according to claim 1, wherein a length of the surgical constricting device is between about 1.75 inches and about 2.5 inches.

11. The surgical constricting device according to claim 1, wherein the elongated body portion is generally cylindrical.

12. The surgical constricting device according to claim 11, wherein each of the first grasping pad and the second grasping pad is in the shape of a rectangular prism.

13. A surgical constricting device, comprising:
an elongated body including a central core and an outer sheath surrounding the central core, the central core made from stainless steel, the outer sheath made from a second material selected from the group consisting of polyurethane, polydimethylsiloxane, polypropylene, polyethylene, and a medium durometer silicone,
wherein the elongated body is configured to maintain its position when wrapped around itself.

14. The surgical constricting device according to claim 13, wherein the second material is a medium durometer silicone.

15. The surgical constricting device according to claim 13, wherein the central core defines a circular cross-section.

16. The surgical constricting device according to claim 15, wherein the circular cross-section of the central core defines a diameter of between about 0.035 inches and about 0.065 inches.

17. The surgical constricting device according to claim 16, wherein the outer sheath defines a thickness of between about 0.014 inches and about 0.045 inches.

18. A method of fixing a catheter to a vessel, comprising:
positioning a surgical constricting device adjacent the vessel such that a central portion of an elongated body of the surgical constricting device is positioned distally of the vessel, and a first end and a second end of the surgical constricting device are positioned proximally of the vessel;
grasping the first end and the second end of the surgical constricting device with an end effector of a surgical grasping instrument; and
rotating the end effector about a longitudinal axis defined by a shaft of the surgical grasping instrument relative to the vessel to create a wrapped portion of the surgical constricting device.

19. The method according to claim 18, wherein rotating the end effector includes rotating the end effector more than 360°.

20. The method according to claim 18, further including removing the surgical grasping instrument from engagement with the surgical constricting device without impacting the wrapped portion of the surgical constricting device.
